(19) United States
(12) Patent Application Publication
(10) Pub. No.: US 2002/0146829 A1
(43) Pub. Date: Oct. 10, 2002

(54) Neutral and anionic colloidal particles for gene delivery

(75) Inventors: Hans Hofland, Foster City, CA (US); Donald Lamons, Emerald Hills, CA (US); Xiao-Ying Meng, Freemont, CA (US)

Correspondence Address: Synnestvedt & Lechner LLP, 2600 Aramark Tower, 1101 Market Street, Philadelphia, PA, 19107-2950, US

(73) Assignee: Aventis Pharmaceuticals Products Inc.

(21) Appl. No.: 09/996,838

(22) Filed: Nov. 29, 2001

Related U.S. Application Data
(60) Provisional application No. 60/253,827, filed on Nov. 29, 2000.

(51) Int. Cl. A61K 48/00; C12N 15/88

(52) U.S. Cl. 435/458; 514/44

(57) ABSTRACT

Processes for making neutral or anionic complexes containing sequestered DNA for gene transfer, by forming a stable colloid containing an aqueous phase having suspended therein a first DNA complex with a cationic surface potential comprising a DNA sequence complexed with a cationic lipid or polymer, and modifying the surface potential of the first DNA complex to form a stable colloid comprising a second DNA complex with a neutral or net anionic surface potential.

### CROSS REFERENCE TO RELATED APPLICATIONS

This application claims priority to and claims the benefit of U.S. provisional application under 35 U.S.C. Section 119(e), 60/253,827, filed Nov. 29, 2000, the disclosure of which is incorporated herein by reference in its entirety.

### BACKGROUND OF THE INVENTION

1. Field of the Invention

The invention provides methods and compositions for small, neutral or negatively charged colloidal structures that enable in vivo targeted DNA delivery to specific cell types.

2. Background information

Gene transfer vehicles, which have been successfully demonstrated to deliver exogenous genes in vivo can be divided into two major groups: the viral and the non-viral vectors, each with their own specific advantages and disadvantages. Non-viral carriers have gained recent attention because they offer attractive features that may overcome problems associated with use of viruses especially in the areas of production and immunogenicity. A wide variety of non-viral gene delivery systems have been described. Lipoplex, polyplex, or lipopolyplex have been described as the most efficient non-viral DNA delivery vehicles for systemic applications. Generally, these complexes contain cationic compounds (i.e. lipids or polymers) that are associated with the anionic DNA through electrostatic interactions, thus condensing and packaging the DNA in colloids that generally have an overall positive charge. The packaging of DNA into these complexes is very efficient. The particles provide protection for the DNA from degradation by nucleases on the one hand, while providing a means for cell uptake on the other. Cell uptake is mediated by binding of these cationic particles to the glycosaminoglycans (esp. heparan sulfates) on the cell surface. This determines the route of entry into the cell that ultimately leads to gene expression.

In addition to these advantages there are some issues associated with the use of non-viral vectors, mostly related to their relatively poor gene transfer in vivo. This lack of activity is due to the cationic nature of the system. After injection, the positive charge of the complex causes the particles to bind anionic proteins present in the circulation. This causes the particles to be opsonized rapidly and subsequently scavenged by the reticulo endothelial system (RES). Recognition of bacterial DNA, especially the CpG motifs, by macrophages of the RES causes production of high levels of cytokines, such as IFN-γ, TNF-α, IL12, the major cause of acute toxicity which is dose limiting.

It would be advantageous to have the possibility to target gene delivery to specific cells. By redirecting the complex away from the RES and towards the target tissue, toxicity will be decreased while increasing efficacy. Unfortunately, the effect of targeting ligands is greatly overshadowed by this non-specific charge induced gene transfer process. In addition, when the positive charges are avoided, e.g. by using conventional liposomes, major problems in DNA packaging efficiency or colloid stability occur.

In the prior art polymers have been used to avoid RES uptake of liposomes. These polymers enable liposomes to circulate for prolonged periods of time and enable them to be targeted once a targeting ligand is conjugated to the end of the polymer chain. Various polymers have been used to sterically stabilize colloidal particles for in vivo use. A typical application is the attachment of polyethylene glycol (PEG) to the surface of liposomes using a lipid anchor (so-called “Stealth” technology)(See Lasic et al., Stealth Liposomes, CRC Press, 1995). This produces a hydrophilic layer on the surface of the liposome, which serves to physically block interactions with other surfaces, i.e. steric stabilization. This layer will reduce the zeta potential of the particle by physically moving the shear plane away from the surface. It does not, however, block the effect of the surface charge beyond the shear plane and out into the solution, as discussed in Fitch, Polymer Colloids, A Comprehensive Introduction, Section 7.4 , Academic Press, 1997. Briefly: when ionized particles move in an electric field, they carry ions with them. The layer of carried ions adds to the hydrodynamic diameter of the particle and defines a new boundary called the shear plane. Past the shear plane, ions and solvent will not be carried by the particle. Elastic light scattering (ELS) can be used to measure the mobility of charged particles in an applied electric field and from that the electrostatic potential (Ψ) at the shear plane is calculated. The magnitude of the electrostatic potential at the shear plane is called the zeta potential (ζ). Surface associated polymers (like PEG) physically control the movement of the solution near the surface and extend the effective shear plane. The zeta potential is reduced without affecting the electrostatic field. This effect is illustrated graphically in FIGS. 1 and 2. The graph shows that steric interactions between particles are very strong, but are effective only close to the surface (dashed line). Electrostatic forces can extend beyond the range of typical surface associated polymers (solid line). That is, a neutral polymer attached at the surface of the particle provides a short-range barrier to collisions, but if the particle carries a strong surface charge, then the charge will be measurable beyond the extent of the surface attached polymer. Therefore, PEG attached to the surface of a cationic complex will only block some of the charge, and the complex will still be cationic to the solution (FIG. 2). Moreover, proteins in solution will continue to be attracted to the field that extents beyond the surface of the polymer, i.e. PEG containing particles will still be opsonized and removed by RES.

### SUMMARY OF THE INVENTION

The present invention provides methods and compositions for efficient DNA packaging in neutral or negatively charged colloids. In addition, this invention enables targeted delivery of DNA to a specific site in the body after systemic administration.

Specifically, the present invention provides methods to reduce, eliminate or reverse the positive charge of particles obtained by complexation of a cationic component to the anionic DNA. The invention also contemplates the composition of the final colloid that enables targeted gene delivery. Preferred methods and compositions of the present invention provide advantages of efficient DNA packaging combined with particle stability in serum and targetability.

Therefore, according to one aspect of the present invention, a process for making neutral or anionic complexes containing sequestered DNA for gene transfer is provided, in which a stable colloid is formed wherein the aqueous phase has suspended therein a first DNA complex with a cationic surface potential containing a DNA sequence complexed with a cationic lipid or polymer, after which the surface potential of the first DNA complex is modified to form a stable colloid containing a second DNA complex with a neutral or net anionic surface potential.

In one embodiment, plasmid DNA is associated with a polyvalent cationic lipid resulting in a stable colloid with a positive surface potential. Preferred lipids contain polyvalent cationic head groups such as linear polyamines (e.g. spermine, spermidine), branched polyamines (RPR209120 cf FIG. 4) or polyamines containing guanidinium groups (RPR204014 cf FIG. 7). In addition, preferred lipids contain hydrophobic moieties that are based on one or more acyl chains of various lengths such as meristyl or palmityl. This colloid is then modified by adding an agent that reacts with the positively charged amines of the cationic lipids, thus consuming the charge of the colloid. Two preferred chemical reagents are citraconic anhydride (CCA) and NHS-Acetate.

In another embodiment, the charge of the complex may be reduced to the point where the colloidal stability is affected. In this case a polymer, such as polyethylene glycol (PEG), may be added to the solution or incorporated into the surface of the particles to replace electrostatic stabilization with steric stabilization.

In yet another embodiment, targeting ligands are added to the complex, either before or after charge modification. These targeting ligands provide an uptake mechanism that provides a route of entry into the cell and ultimately lead to gene expression. Preferred targeting ligands are folate (RPR258018 cf FIG. 13), or tumor homing peptides such as RGD or NGR.

In another application, the charge modification is reversible under acidic pH. Thus triggering the release of the modifying group and reconstituting the highly active cationic complex as is the case for CCA modified RPR204014/DNA complexes (FIG. 7).

The invention will be especially useful for systemic, targeted delivery of DNA. One preferred application is for the treatment of malignant tumors. Small neutral or anionic particles can exit the circulation and enter tumor tissues by means of the well-known enhanced permeation “leaky vasculature” and retention “no lymphatic drainage” (EPR) phenomenon associated with malignant tumors. Other particularly suitable tissues to target include sites of inflammation, liver and spleen. Particles, which are additionally equipped with surface associated ligands, can specifically transfect additional target tissues, such as proliferating endothelial cells.

Non-specific gene transfer and CpG mediated toxicity are the main causes of inefficiency of non-viral gene delivery systems to date. This current invention reduces both non-specific gene transfer and CpG mediated toxicity, enabling cell specific, receptor mediated gene transfer.

Attempts have been made to use neutral or anionic materials to package DNA. However, literature in the field clearly indicates that charge neutrality leads to major problems in packaging efficiency and/or colloid stability. The novelty of this invention lays in the fact that cationic lipids are used to package DNA in their highly effective manner first. Subsequently, the cation is modified into a neutral or anionic compound while retaining DNA packaging and particle stability. Many of the polyvalent lipids with short acyl chains have been shown to give stable particles after chemical modification.

Stable colloids prepared by process of the invention and gene therapy techniques utilizing the colloids are also disclosed. These and other features of the invention and the advantages thereof will become more fully apparent when the following detailed description of the invention is read in conjunction with the accompanying figures.

### BRIEF DESCRIPTION OF THE DRAWINGS

FIG. 1 is a graphical representation of repulsive force versus distance to next surface of like charge as applied to charged particles contained within a colloid. Based on Fitch, Polymer Colloids, A Comprehensive Introduction, Section 7.4, Academic Press, 1997.

FIG. 2 is a graphical and pictorial representation of the variation in electrical potential (Ψ) as a function of distance from the surface.

FIG. 3 is a pictorial representation of charge fields at the surfaces of cationic complexes with and without modification with PEG2000 and/or chemical modification.

FIG. 4 is a representation of the chemical reaction of NHS-acetate with the amine end group(s) of a typical cationic lipid.

FIG. 5 is a graph showing the modification of zeta potential in a cationic lipid complex using NHS-acetate.

FIG. 6 is a graph showing the modification of zeta potential and comparing the reaction of NHS-acetate with different lipid complexes.

FIG. 7 is a representation of the reversible chemical reaction of citraconic acid anhydride with the amine end group of a typical cationic lipid.

FIG. 8 is a composite graph showing biodistribution to all affected tissues using NHS-acetate charge-modified particles made in accordance with Example 1.

FIG. 9 is a composite graph showing biodistribution to all affected tissues using citraconic acid charge-modified particles made in accordance with Example 2

FIG. 10 is a graph of the blood level at 0.5 and 6.0 hours of injected particles of NHS-acetate charge modified particles at different molar ratios of NHS-acetate to lipid.

FIG. 11 is a graph of the percent of total dose in the spleen at 0.5 and 6.0 hours of injected particles of NHS-acetate charge modified particles at different molar ratios of NHS-acetate to lipid.

FIG. 12 is a graph of the percent of total dose in a tumor at 0.5, 6.0 and 24 hours of injected particles of NHS-acetate charge modified particles at different molar ratios of NHS-acetate to lipid.

FIG. 13 Folate mediated gene transfer in vitro of NHS-Acetate modified lipid DNA complex: 1 μg DNA+5 nmoles RPR209120+1nmole RPR204014+0.3 nmole RPR 204293 w/wo 0.3 nmole distearyl-PEG500-Folate (RPR258010), which was inserted after the complex was formed.

FIG. 14 Expression of CAT transgene in different organs after IV injection of 100, 200, 400 or 800 μg DNA in chemically modified particles. Data are mean and individual values of four animals.

### DETAILED DESCRIPTION OF THE INVENTION

The invention provides methods and compositions for DNA packaging in small, neutral or negatively charged colloidal structures that enable in vivo targeted delivery to specific cell types. In accordance with the process of the present invention, complexes are formed comprising a core of plasmid DNA and cationic lipid. These complexes are initially in the form of fine particles suspended in an aqueous environment. The particles initially have a positive surface potential. The colloid is then modified by the addition of an agent that reacts with the positively charged groups of the cationic lipid to reduce, remove or reverse the charge at the surface or throughout the lipid complex. Modifying the charge of these lipid particles reduces their interactions with anionic proteins and cell surfaces to which they are exposed, thus enabling targeted gene delivery.

Stable particles containing active plasmid DNA can be produced using a self-assembling process where cationic lipids or cationic lipid/neutral lipid mixtures are attached to DNA by ionic interactions. The lipids are first put in aqueous suspension as micelles or liposomes. As these particles bind to DNA, a spontaneous rearrangement produces sections of lipids in bilayers sandwiching the DNA. If enough lipid is used, all of the DNA becomes sequestered within the lipid structure and is unavailable to compounds in solution such as DNAse. The particles can be subsequently used for in vivo gene therapy. However, strong positive charge of the particles causes serum proteins to bind, which in turn leads to opsinization and rapid scavenging by the RES system. Uptake and digestion by macrophages causes acute elevation of cytokines, such as IFN-γ, TNF-α or IL12, which leads to a dose limiting toxicity. The process of the present invention takes advantage of this self-assembling system to package the DNA, but adds a chemical modification of the lipids to reduce or reverse the zeta potential. The DNA remains in its convenient protective package, that is, enveloped in lipids. The result is that a cationic lipid is used to package DNA in a complex, which is subsequently altered to form a neutral or anionic colloid in which the DNA is still present. These modified particles have a decreased acute toxicity profile, and are now enabled for the use in targeting approaches by eliminating non-specific charge mediated gene transfer.

Cationic lipids suitable for use in such particles may have primary, secondary or guanidino amines as described, for example, in “Liposomes in Gene Delivery.” D. D. Lasic (Ed.) CRC Press, Boca Raton (1997). Other cationic agents used to condense DNA could be used in this manner. Polymers such as polyethyleneimine, polylysine and dendrimeres are examples.

The cationic lipid particles of the present invention may comprise cationic lipids in combination with neutral lipids. Neutral lipids suitable for use in forming the cationic lipid particles include DOPE, DOPC, Cholesterol, RPR204293 as described, for example, in “Liposomes in Gene Delivery.” D. D. Lasic (Ed.) CRC Press, Boca Raton (1997).

Obvious to one skilled in the art, the choice of solvent is irrelevant to this application.

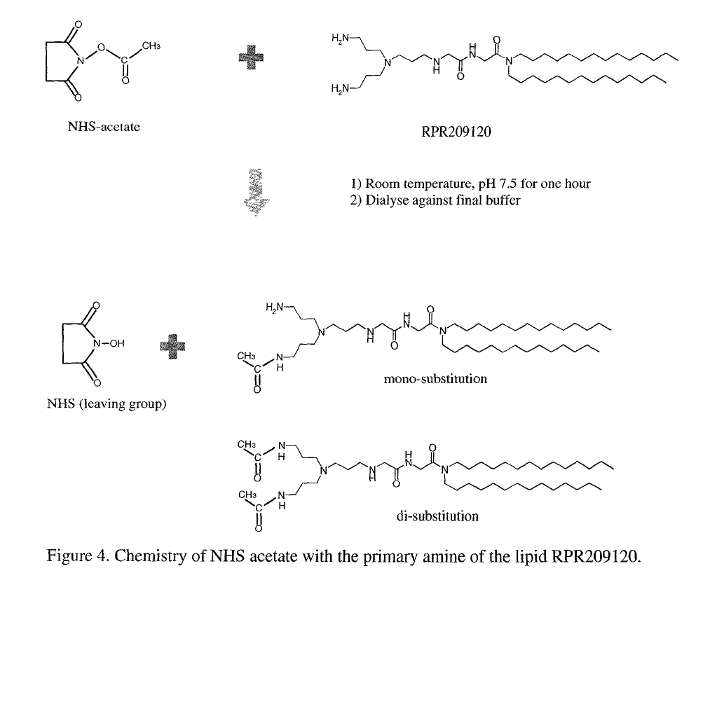

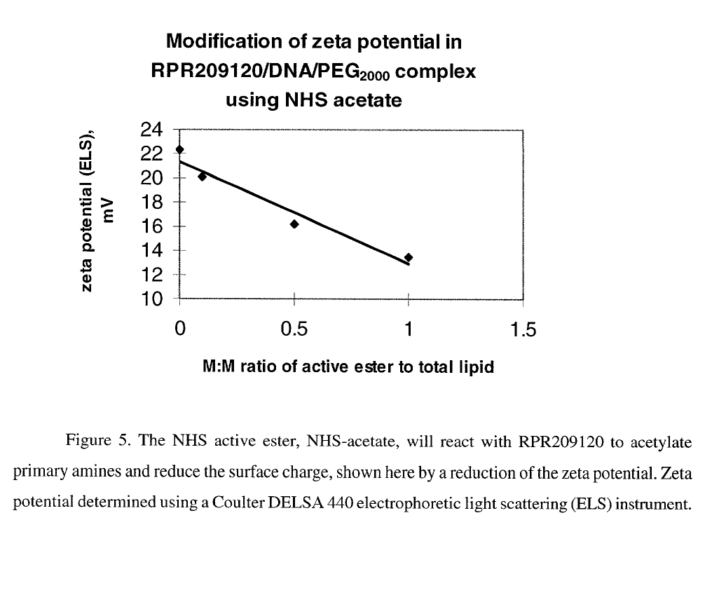

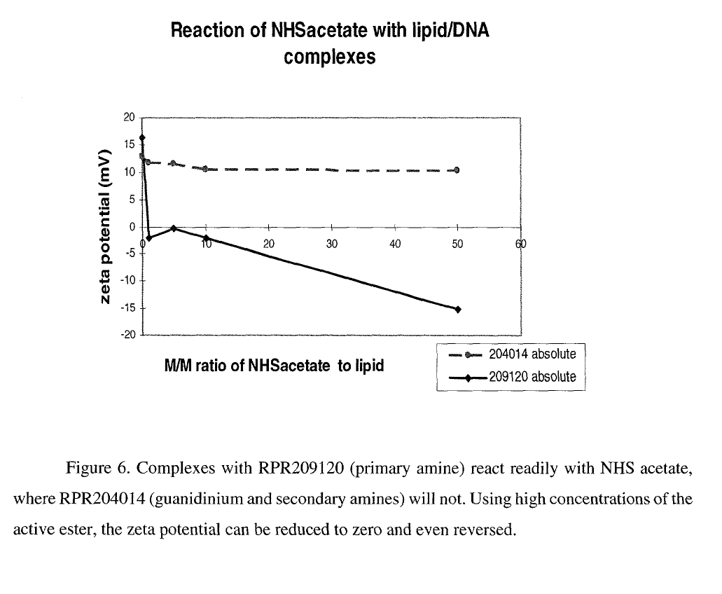

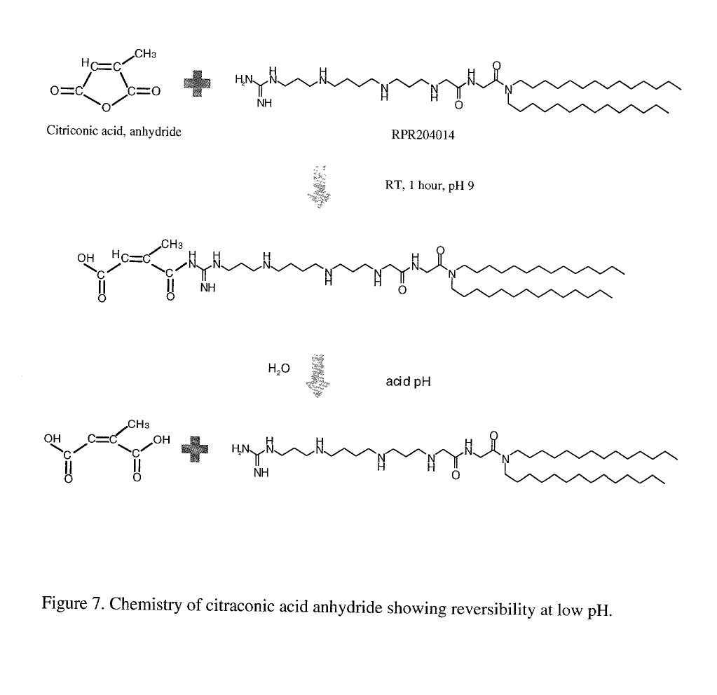

One example of a class of compounds used to react with primary amines is NHS esters. An example of such an ester is the acetic acid derivative: N-hydroxysuccinimide acetate (NHS-acetate). NHS-acetate is a simple blocking reagent and the reaction product is not reversible. The chemical modification of a typical cationic lipid with NHS-acetate is illustrated in FIG. 4. In this example the cationic lipid is RPR209120 (2-(3-[Bis-(3-amino-propyl)-amino]-propylamino)-N-ditetradecylcarbamoylmethyl-acetamide), which has two primary amino groups. The reaction can be mono-substitution to reduce the positive charge or di-substitution to eliminate the positive charge, or a combination thereof to reduce the charge to an intermediate level. FIG. 5 is a graph showing the modification of zeta potential in RPR209120/DNA/PEG2000 complex using NHS-acetate. The data is presented as the zeta potential (ELS) in mV as a function of the molar ratio (M/M) of active ester to total lipid. FIG. 6 is a graph showing the effect on zeta potential for the reaction of NHS-acetate with similar lipid/DNA/PEG2000 complexes using RPR209120 (primary amine) and RPR204014 (a guanidino amine, as shown in FIG. 7). The NHS-acetate reacts readily with the primary amine, but is not as reactive with secondary and guanidino amines such as the RPR204014.

Another preferred class of compounds for use in the charge modification process of the present invention is anhydrides that are reactive with guanidino amine groups. An example of such an anhydride is citraconic acid anhydride (CCA). CCA is reactive with guanidino amines and its chemistry is reversible. The chemical modification of a typical cationic lipid with citraconic acid anhydride is illustrated in FIG. 7. In this example the cationic lipid is RPR204014 (N-Ditetradecylcarbamoylmethyl-2-{3-[4-(3-guanidino-propylamino)-butyl-amino]-propylamino}-acetamide that has a guanidino amine group. In this case the reaction blocks the amine and adds a carboxylic acid functional group. As also shown in FIG. 7, the reaction of citraconic acid anhydride is reversible at low pH, wherein citraconic acid is produced and the lipid returns to its positively charge amide form. This feature of reversibility of the reaction of citraconic acid anhydride at low pH adds a useful adjustability to this reaction.

In cases in which the charge is reduced to the point where the colloid stability is affected, a polymer or other agent may be added to the solution or incorporated into the surface of the particle to replace electrostatic colloid stabilization with steric stabilization.

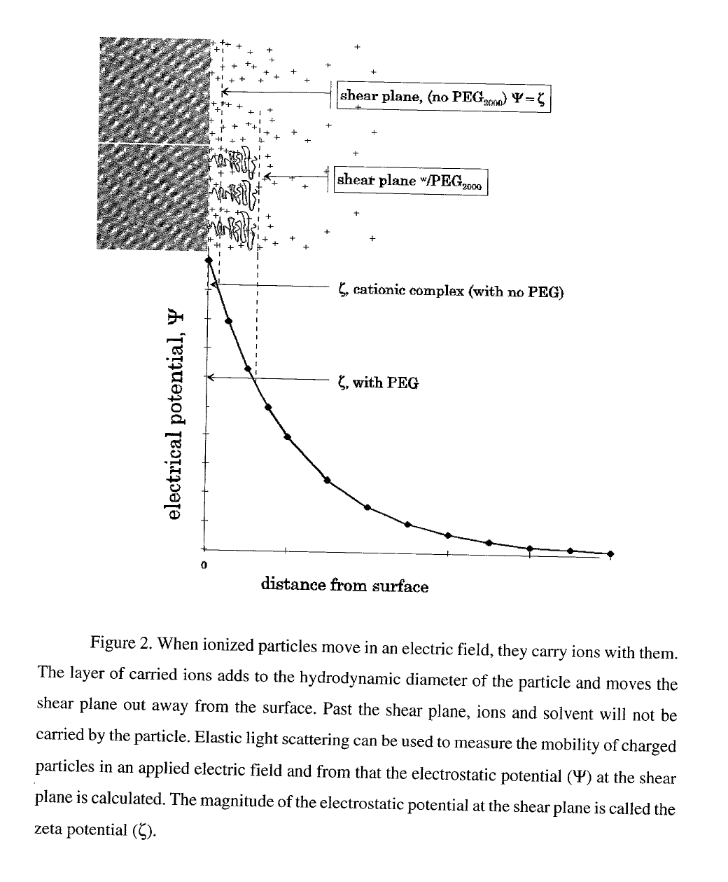

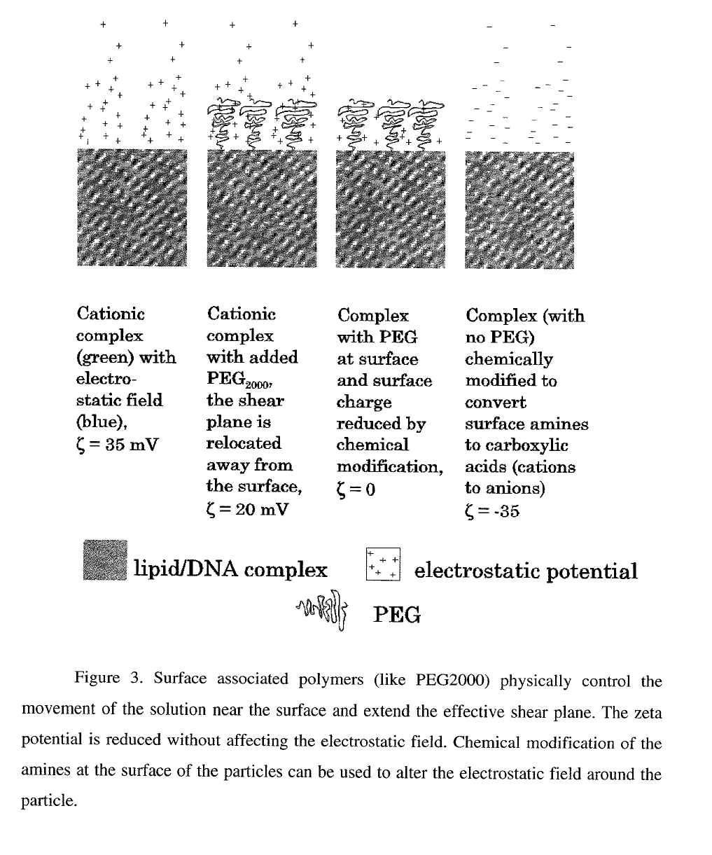

The charge-modification process of the present invention can be applied to sterically protected (i.e. PEG-coated) particles to reduce the surface potential to zero or to even produce anionic particles, as illustrated in FIG. 2, and in greater detail in FIG. 3.

Preferred conditions for these charge modification reactions are those allowing the reaction to take place under mild conditions as to not interfere with particle stability, and/or DNA integrity. More specifically conditions that cause aggregation of the cation/DNA complex should be avoided, i.e. conditions of high ionic strength (>150 mM NaCl). In addition, conditions should be avoided that could damage the DNA integrity, i.e. conditions of extremely high or low pH. Preferred reaction conditions take place in a pH range of 6-8. Also, reactions generating extreme heat should be avoided for the same reasons.

The process of the present invention is particularly useful for particles containing plasmid DNA. Oligonucleotides, RNA and small oligopeptides are also suitable for use in this process. The main objective of the invention is for systemic, targeted delivery of these compounds in vivo. One preferred application is for the treatment of malignant tumors. Other particularly suitable tissues to target include sites of inflammation, liver and spleen. Particles, which are additionally equipped with surface associated ligands, can specifically transfect additional target tissues, such as proliferating endothelial cells, in the absence of strong charge-charge interactions.

The following examples illustrate methods of preparation of charge modified lipid/DNA complexes, which provide efficient packaging of DNA, stable colloidal structures with neutral or negative charge, that are targetable. The examples are intended only to illustrate specific compositions and methods of the invention, but are in no way intended to limit the scope thereof.

### EXAMPLE 1

### NHS-Acetate Modified Complex

This example demonstrates the use of NHS-acetate to modify the surface charge of cationic lipid particles containing plasmid DNA. Cationic particles were made by first combining the cationic lipid RPR209120 (2-(3-[Bis-(3-amino-propyl)-amino]-propylamino)-N-ditetradecylcarbamoylmethyl-acetamide), a typical plasmid DNA and a conjugate of polyethylene glycol and lipid (DSPE-PEG, 1,2-distearoyl-sn-glycero-3-phosphoethanolamine-N-[poly(ethylene glycol) 2000]) (Avanti Polar Lipids, PN 880120). The resulting complex was then reacted with the active ester NHS-acetate.

The lipid components, RPR209120 and DSPE-PEG were combined in chloroform at a molar ratio of 10:1 (10% DSPE-PEG). The lipid components were deposited as a thin film by evaporation of the organic solvent by rotating the solution under a stream of argon or under reduced pressure. The lipid component was further dried under vacuum (less than 0.10 mm Hg for four hours). 5% dextrose, 20 mM NaCl solution was then added to produce a lipid suspension with a concentration of 3 mM. This was incubated over night at 4° C. and then sonicated to produce a uniform suspension of micelles. Equal volumes of this suspension and a plasmid DNA solution (0.5 mg/ml in 5% dextrose, 20 mM NaCl) were quickly mixed to produce a stable colloid of lipid/DNA/PEG complex particles of 70 to 100 nm in diameter (ELS).

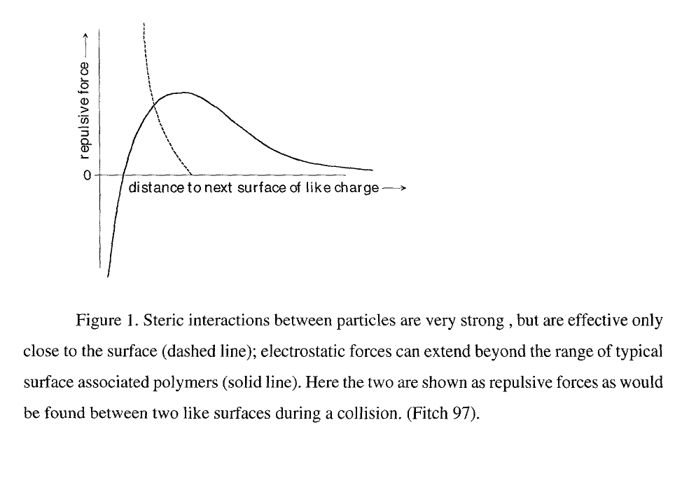

The colloidal suspension of lipid complex was then reacted with a freshly made aqueous solution of an active ester comprising acetic acid and N-hydroxysulfosuccinimide ester (NHS-acetate) at room temperature for one hour and then dialyzed over night at 4° C. against 5% dextrose, 20 mM NaCl. The reaction chemistry is illustrated in FIG. 1. The amount of NHS-acetate active ester can be varied from about one-tenth to about ten times molar ratio to total lipid to produce particles with reduced to no surface charge. Higher ratios of NHS-acetate active ester to total lipid have produced negatively charged particles.

The attached graphs (FIGS. 5 and 6), show the relationship of the amount of NHS-acetate active ester to the resulting zeta potential (ELS) in mV. Particle size by dynamic light scattering analysis increases slightly with the amount of reaction as does the fluorescence signal of ethidium bromide (EB) staining (Table 1). However, DNA was found to be still packaged in the lipid particles, as determined by its retardation during agarose gel electrophoresis.

TABLE 1

| mole | zeta | | |
|---|---|---|---|
| ratio | potential | EB | Size |
| 0 | 16.42 | 4.6 | 131 |
| 1:1 | −2.19 | 5.7 | 151 |
| 5:1 | −0.36 | 10.2 | 168 |
| 10:1 | −2.09 | 11.0 | 169 |
| 50:1 | −15.0 | 13.1 | 189 |

### EXAMPLE 2

### Citraconic Acid Anhydride Modified Complex

Cationic lipid particles consisting of RPR204014 (N-Ditetradecylcarbamoylmethyl-2-{3-[4-(3-guanidino-propylamino)-butylamino]-propylamino}-acetamide and DNA with a ratio of one μg of DNA per 6 nmol of RPR204014 lipid were 60 to 80 nm in diameter with a zeta potential of about 40 mV. Reacting these particles in carbonate buffer, pH 9, with 4 mM citraconic acid anhydride [CAS 616-02-4] (CCA) resulted in stable particles with a zeta potential of about −40 mV. At a lower pH of 5.5 the reaction reverses reverting the particle to cationic. The chemistry of this reaction is illustrated in FIG. 7.

Table 2 presents the zeta potential before and after CCA modification at pH 5.5 for two hours at room temperature (RT).

TABLE 2

| | | Zeta Potential after |
|---|---|---|
| CCA/lipid | Zeta Potential before | incubation at pH 5.5 for |
| Ratio | incubation (mV) | 2 hr at RT (mV) |
| 0 | 11.4 | 20.74 |
| 6 | 9.75 | 12.24 |
| 10 | 0.68 | 5.6 |
| 50 | −6.17 | 7.63 |
| 100 | −10.0 | 8.74 |

### EXAMPLE 3

### Biodistribution Studies

As discussed above, the present process modifies cationic particles to reduce the difference in surface charge between them and the in vivo environment. This reduces the amount of product that is captured onto blood cells and other anionic tissue membranes. Opsonizing proteins may also be inhibited from binding. These particles, being small enough, can then enter certain tissues through openings in the capillary walls, either through fenestrae (as in the liver) or through “leaky vasculature” associated with cancer tumors or inflamed tissues. Such charge-modified particles can then react with cells through weak ionic or entropic processes. Target tissues include sites of inflammation, cancer tumors, tumor endothelium, and liver. Biodistribution in a mouse was tracked using the gamma emitter 111 Indium attached to the complex using a metal chelator-lipid conjugate. The complex was administered by tail vein injection. Mice were pre-injected subcutaneously with cultured tumor cells (4T1) 10 to 14 days prior to testing. The results are set forth as % of injected dose in blood versus molar ratio of NHS-acetate to total lipid, with data shown for circulation times of 30 minutes (squares) and 6 hours (triangles).

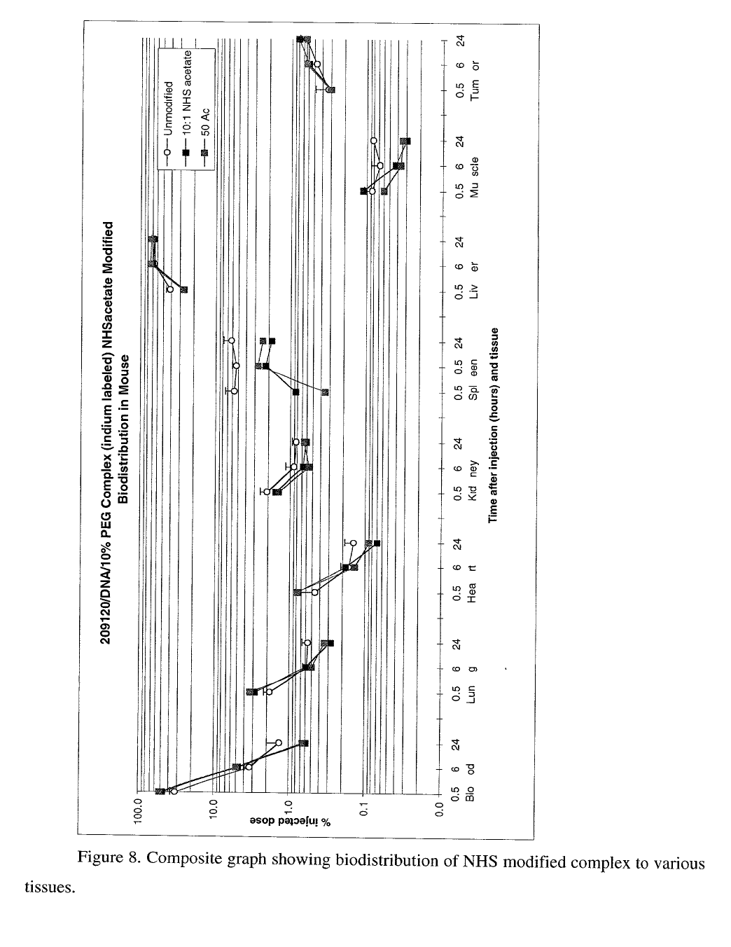

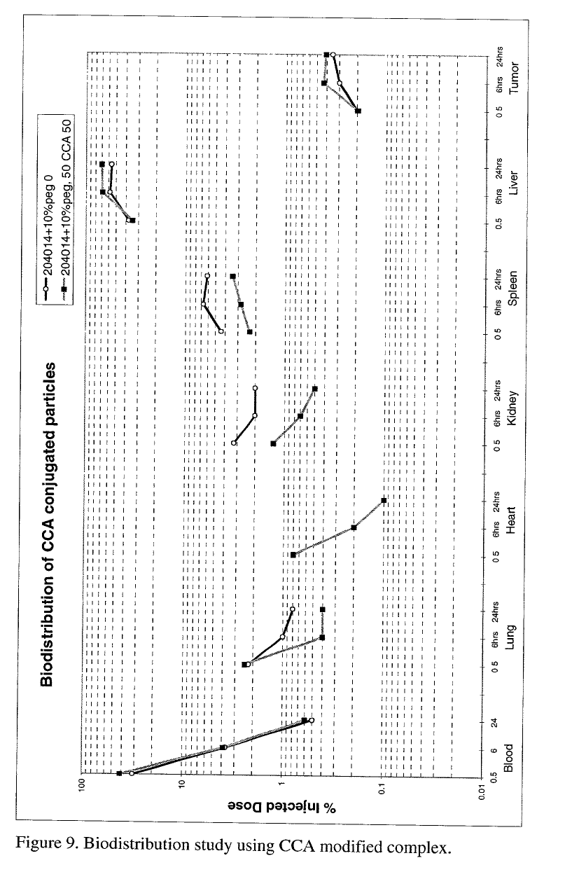

The biodistribution of these particles was measurably different from unmodified particles in circulation time, tumor uptake and affect on spleen. FIG. 8 is a composite graph showing biodistribution to all affected tissues of NHS-acetate charge-modified particles made in accordance with the process of Example 1. FIG. 9 is a composite graph showing biodistribution to all affected tissues of citraconic acid charge-modified particles made in accordance with the process of Example 2.

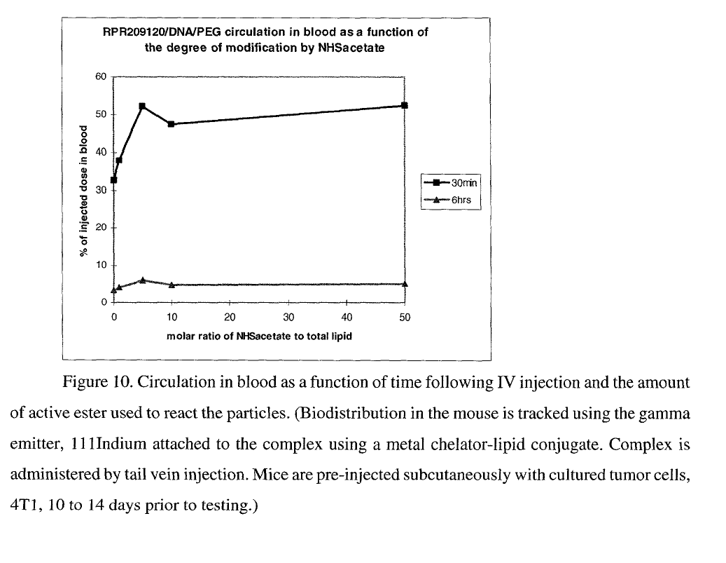

FIG. 10 is a graph of the blood level at 0.5 and 6.0 hours of injected particles of NHS-acetate charge modified particles at different molar ratios of NHS-acetate to lipid. As illustrated in FIG. 10, blood levels were significantly higher for modified particles at both 0.5 and 6 hours. FIG. 10 shows circulation in blood as a function of the degree of modification by NHS-acetate. The data present the time following IV injection of NHS-acetate charge-modified particles made in accordance with the process of Example 1, and the amount of active ester used to react the particles.

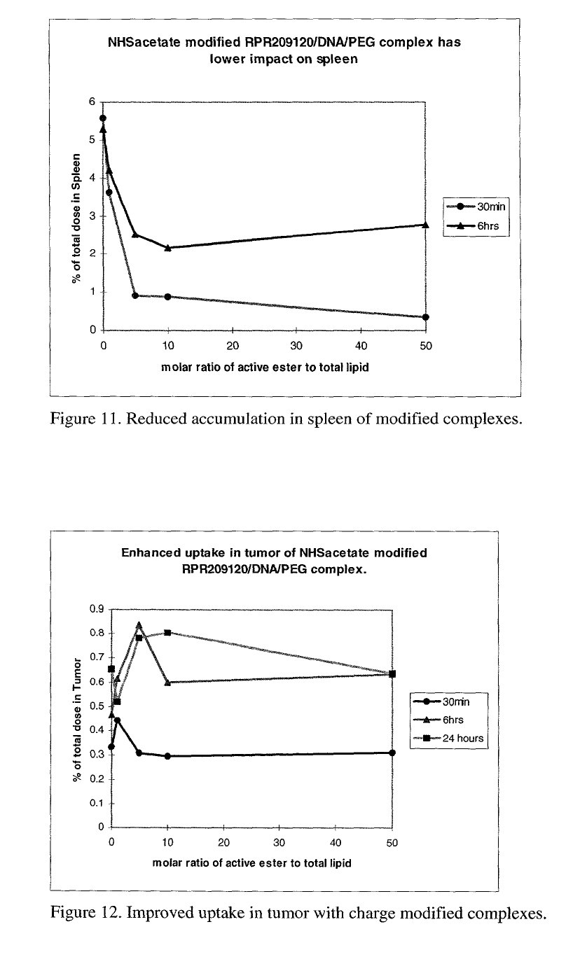

Injected particles should not interact with circulating blood cells. Longer circulation times at 0.5 and 6 hours could be a result of reduced particle adsorption to blood cells. Red blood cells damaged by particles would be expected to end up in the spleen. FIG. 11 presents data, which demonstrate that charge-modified particles have a lower impact on the spleen. The data is set forth as the % of total dose in spleen versus the molar ratio of active ester to total lipid, with data shown for circulation times of 30 minutes (squares) and 6 hours (triangles). Data for the spleen show that with charge modified particles made in accordance with the process of Example 1, less of the trace goes to the spleen.

FIG. 12 shows an enhanced uptake in a tumor of the NHS-acetate modified complex particles of Example 1. The data is set forth as the % of total dose in the tumor versus molar ratio of active ester to total lipid, with data shown for circulation times of 30 minutes (circles), 6 hours (triangles) and 24 hours (squares). About 50% more charge modified complex (5 and 10 molar excess of NHS-acetate) ends up in the tumor after both 6 and 24 hours.

### EXAMPLE 4

### Targeted Gene Transfer In Vitro

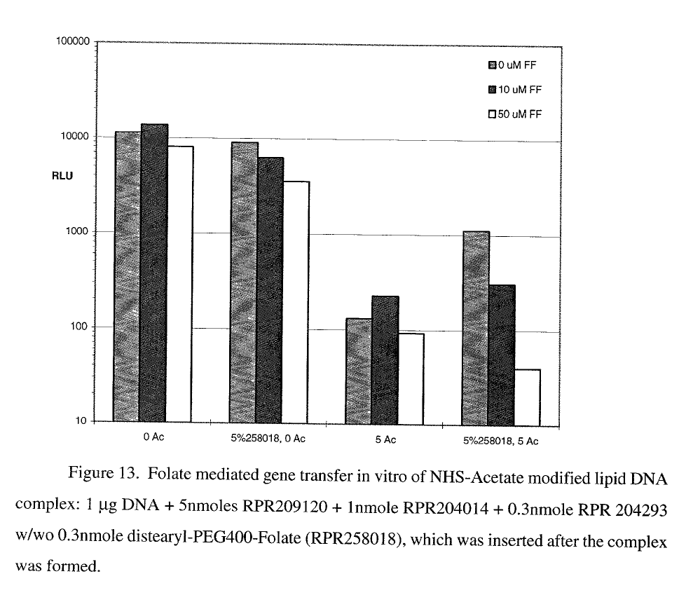

In the absence of strong charge-charge interactions particles which are additionally equipped with surface associated ligands can react with additional targeted tissues, i.e. with cells having binding sites for the specific ligand. The particles contain plasmid DNA which when transfected to the target cells will produce the therapeutic agent over an extended period of time. Folate mediated gene transfer in vitro of NHS-Acetate modified lipid DNA complex was investigated using M109 cells in vitro. The following formulation was used 1 μg DNA+5 nmoles RPR209120+1 nmole RPR204014+0.3 nmole RPR 204293w/wo 0.3 nmole distearyl-PEG400-Folate (RPR258018), which was inserted after the complex was formed. The rationale behind this formulation was: RPR209120 will be modified by NHS-Acetate; RPR204014 will not be modified, i.e. will help stability of the particle, since PEG lipids cannot be used (shielding of targeting ligand containing only short PEG linker); RPR204293 is a neutral helper lipid which will help stabilize the complex and help endosome escape; RPR258018 has been shown to specifically bind to folate receptor on M109 cells. Free Folate (FF) was used to compete with the particles, i.e. to show folate mediated gene transfer. The results are shown in FIG. 13. High nonspecific (not competed with FF) gene transfer was found for non-modified cationic complex with or without RPR258018. Modification of the complex with 5 fold molar excess of NHS-Acetate (vs RPR209120 amines) resulted in a 100-fold reduction in gene transfer. The reduction in gene transfer could be partially (10 fold) restored by addition of the targeting lipid RPR258018. RPR258018 mediated gene transfer of 5Ac-modified complex could be competed with free folate, suggesting receptor mediated gene transfer.

### EXAMPLE 5

### Gene Transfer In Vivo

This example illustrates the use of particles modified with NHS-acetate for in vivo gene transfer. Cationic particles are made by first combining the cationic lipid RPR209120 (2-(3-[Bis-(3 -amino-propyl)-amino]-propylamino)-N-ditetradecylcarbamoylmethyl-acetamide), neutral lipid DOPE (dioleoyl-phosphatidylethanolamine), a conjugate of polyethylene glycol and lipid (DSPE-PEG, 1,2-distearoyl-sn-glycero-3-phosphoethanolamine-N-[poly(ethylene glycol) 2000]) and plasmid DNA encoding chloramphenicol acetyl transferase gene (CAT). The resultant complex is then reacted with the active ester NHS-acetate.

The lipid components, RPR209120, DOPE and DSPE-PEG are combined in chloroform at a molar ratio of 10:10:0.8. The lipid components are deposited as a thin film by evaporation of the organic solvent by rotating the solution under a stream of argon or under reduced pressure. The lipid component is further dried under vacuum (less than 0.10 mm Hg for four hours). 5% dextrose, 20 mM NaCl solution is then added to produce a lipid suspension which is sonicated to produce a uniform suspension of liposomes. Equal volumes of this suspension and a plasmid DNA solution (0.5 mg/ml in 5% dextrose, 20 mM NaCl) are quickly mixed at a ratio of 5 nmoles RPR209120 per microgram of DNA to produce a stable colloid of lipid/DNA/PEG complex particles.

The colloidal suspension of lipid complex is diluted in 100 mM HEPES buffer (pH 7.5), and then reacted with a freshly made aqueous solution of acetic acid N-hydroxysulfosuccinimide ester (NHS-acetate) at room temperature for one hour. The amount of NHS-acetate is five times molar ratio to cationic lipid. Particles are then concentrated by placing PEG 20,000 on top of a dialysis bag. When a final concentration of 0.8 to 1.0 mg DNA/ml is obtained the final product is subsequently dialyzed over night at 4° C. against 5% dextrose, 20 mM NaCl.

Balb-C mice bearing M109 subcutaneous tumors are injected intravenously with increasing amounts of modified particles (corresponding to 100, 200, 400 and 800 μg DNA). Twenty-four hours after injection, mice are sacrificed, main organs are collected, homogenized and the amount of CAT transgene is determined using a standard CAT Elisa (Roche, Ind.).

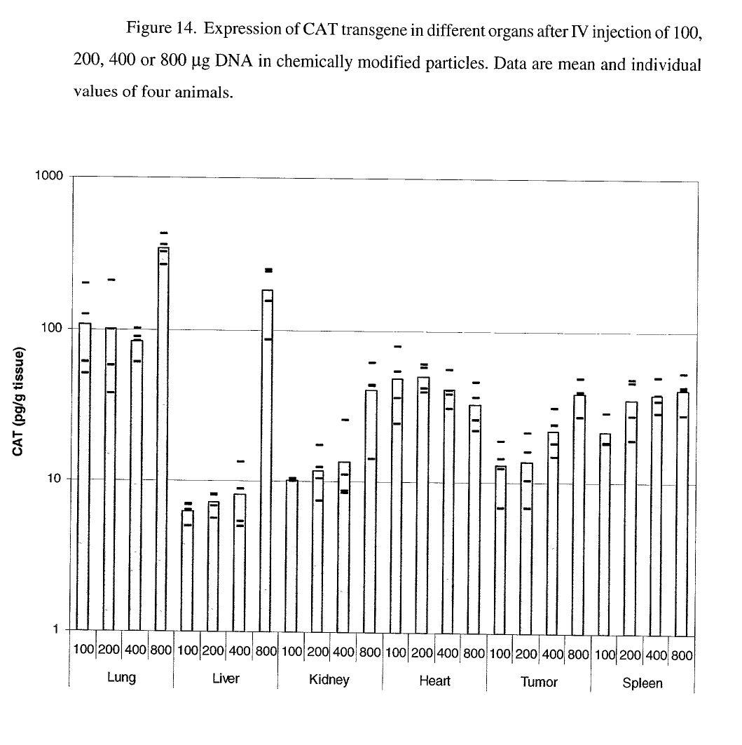

FIG. 14 shows that lung, liver, kidney, heart, spleen and tumor express the CAT transgene.

### EXAMPLE 6

### Characterization of Modified Particles

Particles are formed and modified as described in Example 5. Lipids are then extracted with 25 mM Hepes pH 8.5, 3M NaCl, 1% octylglucoside, dried and analyzed by HPLC with a C4 column using a gradient from 40 to 70% acetonitrile in water in 30 minutes (water and acetonitrile both contain 1% TFA). In these conditions, unmodified RPR209120 has an elution time of 14.8 minutes. Lipid extracted from particles treated with NHS-acetate has an elution time of 19.5 minutes, demonstrating it has been chemically modified by the procedure.

Having thus described a few particular embodiments of the invention, various alterations, modifications, and improvements will readily occur to those skilled in the art. Such alterations, modifications and improvements as are made obvious by this disclosure are intended to be part of this description though not expressly stated herein, and are intended to be within the spirit and scope of the invention. The foregoing description is by way of example only, and not limiting. The invention is limited only as defined in the following claims and equivalents thereto.

What is claimed is:

1. A process for making neutral or anionic complexes containing sequestered DNA for gene transfer, comprising:
forming a stable colloid comprising an aqueous phase having suspended therein a first DNA complex with a cationic surface potential comprising a DNA sequence complexed with a cationic lipid or polymer; and
modifying the surface potential of said first DNA complex to form a stable colloid comprising a second DNA complex with a neutral or net anionic surface potential.

2. The process of claim 1, wherein the surface potential of said first DNA complex is modified by adding a poly(alkylene oxide) to the aqueous phase of said colloid.

3. The process of claim 2, wherein said poly(alkylene oxide) is polyethylene glycol.

4. The process of claim 1, wherein the surface potential of said first DNA complex is modified by the covalent attachment of poly(alkylene oxides) to the cationic lipid or polymer.

5. The process of claim 4, wherein said poly(alkylene oxide) is polyethylene glycol.

6. The process of claim 1, wherein said first DNA complex is a complex of a DNA sequence with a cationic lipid or polymer comprising one or more cationic head groups, and said first DNA complex is modified by reacting said cationic head groups with a reagent that reacts with the cationic head group to neutralize the positive charge thereon.

7. The process of claim 6, wherein said cationic lipid or polymer is selected from the group consisting of linear polyamines, branched polyamines and polyamines comprising guanidinium groups.

8. The process of claim 6, wherein said reagent is citraconic anhydride or N-hydroxysuccinimide acetate.

9. The process of claim 6, wherein reagent is an N-hydroxysuccinimide ester of a targeting ligand, so that a targeting ligand is covalently attached to said cationic lipid or polymer that also modifies the surface potential of said first DNA complex.

10. The process of claim 9, wherein said targeting ligand is an amino sugar or peptide.

11. The process of claim 1, wherein said first DNA complex further comprises a targeting ligand covalently attached to said cationic lipid or polymer.

12. The process of claim 4, wherein said poly(alkylene oxide) is only covalently attached to cationic lipids or polymers on the surface of said first DNA complex.

13. The process of claim 4, wherein said poly(alkylene oxide) is covalently attached to cationic lipids or polymers on the surface of and in the interior of said first DNA complex.

14. The process of claim 6, wherein said reagent is only reacted with cationic head groups of cationic lipids or polymers on the surface of said first DNA complex.

15. The process of claim 6, wherein said reagent is reacted with cationic head groups of cationic lipids or polymers on the surface of and in the interior of said first DNA complex.

16. A stable colloid comprising an aqueous phase having suspended therein a first DNA complex with a cationic surface potential comprising an exogenous therapeutic DNA sequence for delivery in vivo to a patient in need thereof, complexed with a cationic lipid or polymer, wherein said aqueous phase comprises an aqueous solution of a poly(alkylene oxide).

17. A stable colloid comprising an aqueous phase having suspended therein a first DNA complex with a cationic surface potential comprising an exogenous therapeutic DNA sequence for delivery in vivo to a patient in need thereof, complexed with a cationic lipid or polymer, wherein said surface potential of said first DNA complex is modified by the covalent attachment of poly(alkylene oxides) to the cationic lipid or polymer.

18. A stable colloid comprising an aqueous phase having suspended therein a first DNA complex with a cationic surface potential comprising an exogenous therapeutic DNA sequence for delivery in vivo to a patient in need thereof, complexed with a cationic lipid or polymer comprising one or more cationic head groups modified by reaction with a reagent that neutralizes the positive charge thereon.

19. A method for gene therapy by delivering in vivo an exogenous therapeutic DNA sequence to a patient in need thereof comprising administering to said patient an effective amount of the colloid of claim 16, 17 or 18.
